(19) United States
(12) Patent Application Publication
(10) Pub. No.: US 2024/0139207 A1
(43) Pub. Date: May 2, 2024

(54) METHODS FOR TREATING PULMONARY HYPERTENSION

(71) Applicant: United Therapeutics Corporation, Silver Spring, MD (US)

(72) Inventors: Meredith Broderick, Silver Spring, MD (US); Andrew Nelsen, Silver Spring, MD (US)

(73) Assignee: United Therapeutics Corporation, Silver Spring, MD (US)

(21) Appl. No.: 18/497,668

(22) Filed: Oct. 30, 2023

Related U.S. Application Data
(60) Provisional application No. 63/421,111, filed on Oct. 31, 2022.

(51) Int. Cl. A61K 31/5575; A61K 31/27; A61K 31/4965; A61K 31/5585; A61K 45/06; A61P 9/12

(52) U.S. Cl. CPC A61K 31/5575; A61K 31/27; A61K 31/4965; A61K 31/5585; A61K 45/06; A61P 9/12; A61K 9/0053

(57) ABSTRACT

The present disclosure provides for treating pulmonary hypertension by administering to a subject suffering from pulmonary hypertension a therapeutically effective amount of a non-oral therapeutic agent for treating pulmonary hypertension followed by an orally administered form of a therapeutic agent for treating pulmonary hypertension, wherein the amount of non-oral therapeutic agent is sufficient to allow for an increased amount of the orally administered therapeutic agent to thereafter be administered compared to a subject who is not previously treated with the non-oral therapeutic agent.

### CROSS REFERENCE TO RELATED APPLICATIONS

The present application claims priority to U.S. provisional application No. 63/421,111 filed on Oct. 31, 2022, which is incorporated herein by reference in its entirety.

### TECHNICAL FIELD

The present invention relates to methods for treating pulmonary hypertension by first administering to a subject in need thereof a non-oral form of a therapeutic agent for treating pulmonary hypertension followed by an oral form of to therapeutic agent for treating pulmonary hypertension.

### BACKGROUND

All blood is driven through the lungs via the pulmonary circulation in order, among other things, to replenish the oxygen which it dispenses in its passage around the rest of the body via the systemic circulation. The flow through both circulations is in normal circumstances equal, but the resistance offered to it in the pulmonary circulation is generally much less than that of the systemic circulation. When the resistance to pulmonary blood flow increases, the pressure in the circulation is greater for any particular flow. The above-described condition is referred to as pulmonary hypertension (PH). Generally, pulmonary hypertension is defined through observations of pressures above the normal range pertaining in the majority of people residing at the same altitude and engaged in similar activities.

Pulmonary hypertension may occur due to various reasons and the different entities of pulmonary hypertension were classified based on clinical and pathological grounds in 5 categories according to the World Health Organization (WHO) convention, see e.g. Simonneau et al., “Clinical Classification of Pulmonary Hypertension,” J. American College of Cardiology, 2004; 43(12 Suppl S):5S-12S. Pulmonary hypertension can be a manifestation of an obvious or explicable increase in resistance, such as obstruction to blood flow by pulmonary emboli, malfunction of the heart's valves or muscle in handling blood after its passage through the lungs, diminution in pulmonary vessel caliber as a reflex response to alveolar hypoxia due to lung diseases or high altitude, or a mismatch of vascular capacity and essential blood flow, such as shunting of blood in congenital abnormalities or surgical removal of lung tissue. In addition, certain infectious diseases, such as HIV and liver diseases with portal hypertension may cause pulmonary hypertension. Autoimmune disorders, such as collagen vascular diseases, also often lead to pulmonary vascular narrowing and contribute to a significant number of pulmonary hypertension patients. The cases of pulmonary hypertension remain where the cause of the increased resistance is as yet inexplicable are defined as idiopathic (primary) pulmonary hypertension (iPAH) and are diagnosed by and after exclusion of the causes of secondary pulmonary hypertension and are in the majority of cases related to a genetic mutation in the bone morphogenetic protein receptor-2 gene. The cases of idiopathic pulmonary arterial hypertension (PAH) tend to comprise a recognizable entity of about 40% of patients cared for in large specialized pulmonary hypertension centers. Approximately 65% of the most commonly afflicted are female and young adults, though it has occurred in children and patients over 50. Life expectancy from the time of diagnosis is short without specific treatment, about 3 to 5 years, though occasional reports of spontaneous remission and longer survival are to be expected given the nature of the diagnostic process. Generally, however, disease progress is inexorable via syncope and right heart failure and death is quite often sudden.

Pulmonary hypertension refers to a condition associated with an elevation of pulmonary arterial pressure (PAP) over normal levels. In humans, a typical mean PAP is approximately 12-15 mm Hg. Pulmonary hypertension, on the other hand, can be defined as mean PAP above 25 mmHg, assessed by right heart catheter measurement. Pulmonary arterial pressure may reach systemic pressure levels or even exceed these in severe forms of pulmonary hypertension. When the PAP markedly increases due to pulmonary venous congestion, such as in left heart failure or valve dysfunction, plasma can escape from the capillaries into the lung interstitium and alveoli. Fluid buildup in the lung (pulmonary edema) can result, with an associated decrease in lung function that can in some cases be fatal. Pulmonary edema, however, is not a feature of even severe pulmonary hypertension due to pulmonary vascular changes in all other entities of this disease.

Pulmonary hypertension may either be acute or chronic. Acute pulmonary hypertension is often a potentially reversible phenomenon generally attributable to constriction of the smooth muscle of the pulmonary blood vessels, which may be triggered by such conditions as hypoxia (as in high-altitude sickness), acidosis, inflammation, or pulmonary embolism. Chronic pulmonary hypertension is characterized by major structural changes in the pulmonary vasculature, which result in a decreased cross-sectional area of the pulmonary blood vessels. This may be caused by, for example, chronic hypoxia, thromboembolism, collagen vascular diseases, pulmonary hypercirculation due to left-to-right shunt, HIV infection, portal hypertension or a combination of genetic mutation and unknown causes as in idiopathic pulmonary arterial hypertension.

Multiple agents can treat pulmonary hypertension including, prostacyclin, prostacyclin analogs and prostacyclin receptor agonists. See Mandras et al., “Combination Therapy in Pulmonary Arterial Hypertension—Targeting the Nitric Oxide and Prostacyclin Pathways,” J. Cardiovascular Pharmacology Theory, 2021 Sept; 26(5). See also Gomberg-Maitland and Olschewski, “Prostacyclin therapies for the treatment of pulmonary arterial hypertension,” European Respiratory Journal, 2008; 31. One such prostacyclin analog is treprostinil. Treprostinil is approved for treating pulmonary hypertension in intravenous and subcutaneous form as Remodulin® and in orally administered form as Orenitram®. One such prostacyclin receptor agonist is ralinepag.

Pulmonary hypertension patients who attained higher total daily doses of oral treprostinil had better outcomes, including increased 6-minute walk distances. See Balasubramanian et al., “Dosing characteristics of oral treprostinil in real-world clinical practice,” Pulmonary Circulation, 2018 Apr-Jun; 8(2): 2045894018770654. See also Ramani et al., “Novel dose— response analyses of treprostinil in pulmonary arterial hypertension and its effects on six-minute walk distance and hospitalizations,” Pulmonary Circulation. 2020; 10(3).

Pulmonary arterial hypertension patients treated with ralinepag had better outcomes, including pulmonary vascular resistance and 6-minute walk distances, than those treated with a placebo. See Torres et al., “Efficacy and safety of ralinepag, a novel oral IP agonist, in PAH patients on mono or dual background therapy: results from a phase 2 randomised, parallel group, placebo-controlled trial,” European Respiratory Journal, 2019; 54: 1901030.

### SUMMARY OF THE INVENTION

One embodiment is a method for treating pulmonary hypertension comprising administering to a subject suffering from pulmonary hypertension a therapeutically effective amount of a non-oral form of a therapeutic agent for treating pulmonary hypertension followed by administering an oral form of a therapeutic agent for treating pulmonary hypertension, wherein the therapeutically effective amount of the non-oral form of the therapeutic agent is sufficient to allow for an increased amount of the orally administered the therapeutic agent to thereafter be administered as compared to a subject who was not previously treated with the non-oral therapeutic agent. In addition to achieving a higher total daily dose of the oral therapeutic agent, a subject treated according to this embodiment may also experience improvement in at least one side effect selected from the group consisting of headache, nausea, and vomiting. In some embodiments, the non-oral therapeutic agent is an inhaled therapeutic agent or a parenteral agent. In some embodiments, the subject is a human.

### BRIEF DESCRIPTION OF THE DRAWINGS

FIGS. 1A-1D show clinical parameters at baseline and Week 16. FIG. 1A) WHO FC is presented as the percentage of patients in each class at baseline and Week 16; the p-value for improvement in function class corresponds to the median individual change from baseline and was obtained using McNemar's test. The total percentage at Week 16 adds up to 99% as percentages were rounded down to the nearest whole integer. FIG. 1B) NT-proBNP, FIG. 1C) 6MWD and FIG. 1D) RA area, are presented as medians with boxes representing interquartile range. P-values in FIGS. 1B-1D correspond to median individual change from baseline and were obtained using Wilcoxon signed-rank test. WHO FC: World Health Organization Functional Class; NT-proBNP: N-terminal-pro brain natriuretic peptide; 6MWD: 6-minute walk distance; RA: right atrial.

FIGS. 2A-2D show risk stratification at baseline and Week 16. Risk strata are based on the 2015 ESC/ERS Guidelines. Each graph represents the percentage of patients in each risk strata at baseline and Week 16 for FIG. 2A) WHO FC, FIG. 2B) NT-proBNP, FIG. 2C) 6MWD, and FIG. 2D) RA area. Of the 29 patients in the per-protocol population, only those with baseline and Week 16 assessments are presented here. WHO FC: World Health Organization Functional Class; NT-proBNP: N-terminal-pro brain natriuretic peptide; 6MWD: 6-minute walk distance; RA: right atrial.

### DETAILED DESCRIPTION

Unless otherwise specified, “a” or “an” means “one or more.” In one aspect, provided herein are methods for treating a subject suffering from a condition associated with an elevated blood pressure.

The term “subject” as used herein refers to living multi-cellular organisms, including vertebrate organisms, a category that include both human and non-human mammals. The methods and compositions as disclosed herein have equal application in medical and veterinary settings. Thus, the general term “subject” under the treatment is understood to include all animals, such as humans, domestic animals, wild animals and laboratory animals.

The term “treating” or “treatment” covers the treatment of a disease or disorder described herein, in a subject, such as a human, and includes: (i) inhibiting a disease or disorder, e.g., arresting its development; (ii) relieving a disease or disorder, e.g., causing regression of the disorder; (iii) slowing progression of the disorder; and/or (iv) inhibiting, relieving, or slowing progression of one or more symptoms of the disease or disorder. For example, treatment of a condition associated with an elevated blood pressure includes but is not limited to, preventing or ameliorating elevation of blood pressure in a subject's pulmonary circulation and/or systemic circulation above the normal range, as well as ensuing symptoms and complications.

The term “pulmonary hypertension” includes patients suffering from pulmonary hypertension associated with or secondary to other conditions, including interstitial lung disease or fibrosis.

A “pharmaceutical agent” or “drug” as used herein refers to a chemical compound or other composition capable of inducing a desired therapeutic or prophylactic effect when properly administered to a subject. In some embodiments, a pharmaceutical agent or drug may be administered as a prodrug, substrate or precursor, which terms refer to a compound that, after administration, is metabolized (i.e., converted within the body) into a pharmacologically active agent. For example, a prodrug, substrate or precursor may be used to improve absorption, distribution, metabolism and/or excretion (ADME scheme) of the corresponding drug, thus improving pharmacodynamics, such as bioavailability, of the corresponding drug.

The term “pharmaceutically acceptable” as used herein refers to safe and sufficiently non-toxic for administration to a subject.

The term “therapeutically effective amount” as used herein refers to a quantity of compound sufficient to achieve a desired effect in a subject being treated. For example, an therapeutically effective amount of a therapeutic agent or drug for treating pulmonary hypertension may be the amount necessary to ameliorate or inhibit elevation of pulmonary arterial pressure over normal levels in the subject, and more particularly, the amount sufficient for maintaining one or more of the right atrial pressure, pulmonary capillary wedge pressure, right ventricular systolic and diastolic pressures, pulmonary artery systolic and diastolic pressures, filling pressure within the normal range, in the subject.

Particularly, contemplated herein are methods and compositions for treating or preventing a condition in a subject associated with elevated blood pressure in the lungs, such as pulmonary hypertension. In some embodiment, this includes treating a subject suffering from neonatal pulmonary hypertension. In other embodiments, this includes treating a subject suffering from primary and/or secondary pulmonary hypertension. In some embodiments, this includes treating a subject suffering from pulmonary arterial hypertension (PAH). The “pulmonary hypertension” treated can be one or more of the WHO classifications for pulmonary hypertension: Group 1 (pulmonary arterial hypertension); Group 1′ (pulmonary veno-occlusive disease (PVOD) and/or pulmonary capillary haemangiomatosis (PCH)); Group 2 (Pulmonary hypertension due to left heart diseases); Group 3 (pulmonary hypertension due to lung diseases and/or hypoxemia); Group 4 (chronic thromboembolic pulmonary hypertension (CTEPH)); Group 5 (pulmonary hypertension with unclear multifactorial mechanisms). For example, “pulmonary hypertension” can refer to any of Group 1-5 pulmonary hypertension or any combination of those groups.

Also contemplated herein are methods and compositions for treating or preventing other conditions associated with elevated blood pressure or decreased blood flow, including vasospasm, stroke, angina, ischemia, revascularization of coronary arteries and other arteries (peripheral vascular disease), transplantation (e.g., of kidney, heart, lung, or liver), treatment of low blood pressure (such as that seen in shock or trauma, surgery and cardiopulmonary arrest) to prevent reperfusion injury to vital organs, cutaneous ulcers (e.g., with topical, non-acidified nitrite salt), Reynaud's phenomenon, treatment of hemolytic conditions (such as sickle cell, malaria, TTP, and HUS), hemolysis caused by immune incompatibility before and after birth, and other conditions.

Treatment of diseases in a subject can be through the administration of suitable pharmaceutical agent(s) or drugs to the subject suffering from the disease or a condition or symptom associated with the disease.

In some embodiments, the non-oral form of therapeutic agent for treating pulmonary hypertension is a prostacyclin (e.g., flolan, treprostinil, beraprost, iloprost), a prostanoid drug, or a prostacyclin receptor agonist such as ralinepag. In some embodiments, the non-oral and the oral therapeutic agent are the same. In an embodiment, the non-oral therapeutic agent is treprostinil and the oral therapeutic agent is treprostinil.

One embodiment of the invention is a method of administering to a subject, such as a human, suffering from pulmonary hypertension a first therapeutically effective amount of a non-oral therapeutic agent for treating pulmonary hypertension followed by an orally administered form of a therapeutic agent for treating pulmonary hypertension, wherein the first therapeutically effective amount of a non-oral therapeutic agent for treating pulmonary hypertension is sufficient to allow for an increased amount of the orally administered therapeutic agent to thereafter be administered compared to a subject who was not previously treated with a non-oral therapeutic agent for treating pulmonary hypertension.

In some embodiments the first therapeutically effective amount of the non-oral therapeutic agent is administered parenterally. In other embodiments, the first therapeutically effective amount of the non-oral therapeutic agent is inhaled by the subject.

Particularly, in some embodiments, the therapeutic agent for treating pulmonary hypertension is treprostinil. In some embodiments, the form of treprostinil used may be a pharmaceutically acceptable salt or ester, or a prodrug of treprostinil. Suitable salts of treprostinil include the sodium, potassium and diethanolamine salts. Other suitable esters and salts of treprostinil are disclosed in U.S. Pat. Nos. 9,278,901 and 9,701,611. Suitable prodrugs of treprostinil are also disclosed in U.S. Ser. Nos. 16/434,938 and 17/001,123, as well as U.S. Pat. No. 9,371,264.

In some embodiments the first therapeutically effective amount of a non-oral therapeutic agent for treating pulmonary hypertension is parenteral treprostinil. More particularly, in some embodiments the parenteral treprostinil is intravenous treprostinil. In other embodiments, the parenteral treprostinil is subcutaneous treprostinil.

One embodiment of the invention is a method of administering to a subject, such as a human, suffering from pulmonary hypertension a therapeutically effective amount of a non-oral prostacyclin analog followed by an orally administered form of a prostacyclin receptor agonist, wherein the therapeutically effective amount of the non-oral prostacyclin analog is sufficient to allow for an increased amount of the orally administered prostacyclin receptor agonist to thereafter be administered compared to a subject who is not previously treated with a prostacyclin analog.

Particularly, in some embodiments, the prostacyclin receptor agonist is ralinepag. In some embodiments, the form of ralinepag used may be a pharmaceutically acceptable salt, or a prodrug of ralinepag. Suitable prodrugs of ralinepag are disclosed in WO 2023/177877. Other suitable forms of ralinepag are also disclosed in WO 2023/158634.

In some embodiments, multiple oral therapeutic agents are co-administered to the subject for treating pulmonary hypertension, in addition to prostacyclin, a prostacyclin analog, or a prostacyclin receptor agonist. The term “co-administer” or “co-administration” as used herein means that multiple therapeutic agents, such as a phosphodiesterase type 5 inhibitor (PDE5) inhibitor, endothelin receptor antagonist, soluble guanylate cyclase stimulator, are administered so that their respective effective periods of biological activity will overlap in the subject being treated. Co-administration can be carried out by contemporaneous or sequential administration of multiple therapeutic agents, for example, administering a second therapeutic agent before, during or after the administration of a first therapeutic agent.

The following example further illustrates though in no way limits the scope of the foregoing embodiments.

### Example 1

### Rapid Parenteral Treprostinil Titration and Transition to Oral Treprostinil

### SUMMARY

A phase 4, multicenter, open-label 16-week study of Remodulin® (intravenous or subcutaneous treprostinil) induction followed by oral Orenitram® (oral treprostinil) optimization in patients with pulmonary arterial hypertension was conducted. Patients could be on other non-prostacyclin class pulmonary arterial hypertension therapies during the course of the study. Enrollment into the study was completed with a total of 35 patients enrolled. Thirty-two patients initiated oral treprostinil. Twenty-nine patients were in the per-protocol population, which included all patients without major protocol deviations. Twenty-eight patients from the per-protocol population completed the study. Once enrolled, patients were initiated on intravenous or subcutaneous Remodulin® in an inpatient or outpatient setting and titrated to a minimum dose of 20 ng/kg/min over two to eight weeks. Patients were then transitioned to Orenitram® over one to 21 days in the inpatient or outpatient setting. The primary endpoint was to evaluate the percentage of subjects achieving an Orenitram® dose of 4 mg three times daily (TID)— or a total daily dose of 12 mg—or higher at Week 16.

The secondary endpoints of this study included measurements in: changes in prostanoid adverse events (AEs), echocardiograms, change in 6-minute walk distance (6MWD), change in Borg dyspnea score, change in World Health Organization (WHO) functional class (FC), change in serum N-terminal pro-brain natriuretic peptide (NT-proBNP) levels, impact of pulmonary hypertension on a person's life (health-related quality of life), and treatment satisfaction. Specifically, this study sought to measure the percentage of subjects that improved in each of the following individual four clinical parameters at Week 16 (6MWD, NT-proBNP, WHO FC, right atrial area) to a lower risk stratum, as defined by the 2015 ESC/ERS guidelines (Gailè et al., DOI: 10.1183/13993003.01032-2015), compared to baseline measurements. An additional endpoint was determining the percentage of subjects that meet each of the following four individual clinical parameters at Week 16 in the low risk category, as defined by 2015 ESC/ERS guidelines: 6MWD>440 meters, serum NT-proBNP.

In this clinical trial, patients enrolled in the study achieved a mean total daily Orenitram® dose of 16.4 mg at 16 weeks with 79% of study subjects reaching the study's primary endpoint of a 12 mg total daily dose. Treatment with Orenitram®, three times daily, was well tolerated and the safety profile was consistent with previous Orenitram® studies in pulmonary arterial hypertension. In the study, several well-known treprostinil adverse events such as headache, nausea, and vomiting tended to improve after transition to Orenitram® (oral treprostinil) from Remodulin® (parenteral treprostinil).

### Methods

Inclusion Criteria

Based on the 2015 ESC/ERS Guidelines, specific hemodynamic inclusion criteria included a mean pulmonary arterial pressure ≥25 mmHg, pulmonary artery wedge pressure or left ventricular end-diastolic pressure ≤15 mmHg, and pulmonary vascular resistance >3 Wood units, in the absence of unrepaired congenital heart disease. Other key inclusion criteria were World Health Organization functional class (FC) II or III, 6-minute walk distance (6MWD)≥250 m, and a REVEAL 2.0 risk score ≤9 (Benza et al. 2021, DOI: https://doi.org/10.1016/j.chest.2020.08.2069). Patients could be receiving up to two oral PAH background therapies if taken at a stable dose for ≥30 days prior to baseline. As this study focused on the addition of a new therapy rather than the replacement of one therapy by another therapy, participants were eligible regardless of the number of pulmonary arterial hypertension background therapies (0, 1, or 2) they were receiving at baseline. Patients were excluded if they had received any prostacyclin-class therapy (i.e., treprostinil, epoprostenol, iloprost, or selexipag) within 28 days of baseline, or had a diagnosis of uncontrolled sleep apnea, renal insufficiency, Child-Pugh B or C hepatic disease, or ischemic heart disease with a pulmonary arterial wedge pressure (PAWP)≥15 mmHg or left ventricular ejection fraction (LVEF)<50%. Baseline assessments were collected up to 14 days prior to initiation of parenteral treprostinil, and included hemodynamics via echocardiogram and RHC, FC, N-terminal-pro brain natriuretic peptide (NT-proBNP), 6MWD, and medication history. Of note, historical RHC data could be used for baseline assessments if performed up to 180 days prior to initiating parenteral treprostinil. Echocardiograms were uploaded and stored in a central repository and evaluated by an independent central reader in accordance with the American Society of Echocardiography Guidelines (Bossone et al., DOI: 10.1016/j.echo.2012.10.009). Table 1 shows selected baseline characteristics for the patients in the per-protocol population.

TABLE 1
Selected characteristics of the 29 patients in the
per-population who received oral treprostinil.
Characteristic

| | | |
|---|---|---|
| Age, years [median (range)] | 56 | (35-72) |
| Female [n (%)] | 18 | (62.1) |

Race [n (%)]

| | | |
|---|---|---|
| White | 23 | (79.3) |
| Black/African American | 4 | (13.8) |
| American Indian/Alaska Native | 1 | (3.4) |
| Other | 1 | (3.4) |
| BMI, kg/m² [median (range)] | 30.8 | (22.5-45.3) |

PAH etiology [n (%)]

| | | |
|---|---|---|
| Idiopathic | 12 | (41.4) |
| CTD- associated | 9 | (31.0) |
| Drug/toxin-associated | 5 | (17.2) |
| HIV-associated | 1 | (3.4) |
| Heritable | 1 | (3.4) |
| Other | 1 | (3.4) |
| Weeks since diagnosis [median (range)] | 33 | (1-274) |

Current PAH therapy [n (%)]

| | | |
|---|---|---|
| PDE-5i or sGCs + ERA | 12 | (41.4) |
| PDE-5i or sGCs alone | 10 | (34.5) |
| ERA alone | 1 | (3.4) |
| None | 6 | (20.7) |

WHO functional class at baseline-no (%)

| | | |
|---|---|---|
| II | 15 | (51.7) |
| III | 14 | (48.3) |
| NT-proBNP-ng/L, median, IQR | 415 | (195-1061) |
| 6MWD-m, median, IQR | 363 | (288-426) |

eGFR-no (%)

| | | |
|---|---|---|
| ≥60 mL/min/1.73 m² | 22 | (75.9) |
| <60 mL/min/1.73 m² | 3 | (10.3) |
| Not available | 4 | (13.8) |
| REVEAL 2.0 risk score-median (IQR) | 7 | (5-8) |

Echocardiographic parameters

| | | |
|---|---|---|
| TAPSE (mm)-median (IQR) | 17.0 | (12.4-21.2) |
| RAA (cm²)-median (IQR) | 20.3 | (16.8-28.9) |
| RV/LV ratio, diastole-median (IQR) | 0.8 | (0.6-0.9) |

Pericardial effusion-no (%)

| | | |
|---|---|---|
| Mild, moderate, or severe | 1 | (3.4) |
| Trace or none | 28 | (96.6) |

Hemodynamics (Right Heart Catheterization)-median
(IQR) (collected in patients up to 180 days screening)

| | | |
|---|---|---|
| mRAP (mmHg) | 7.0 | (4.0-10.0) |
| mPAP (mmHg) | 53 | (47-58) |
| PAWP (mmHg) | 10 | (7-13) |
| PVR (Wood units) | 9.6 | (7.5-11.6) |
| Cardiac index (L/min/m²) | 2.4 | (2.1-2.7) |

Treatment

Patients were initiated on 2 ng/kg/min of subcutaneous (SC) or intravenous (IV) treprostinil in an inpatient or outpatient setting per clinician decision. Parenteral treprostinil was titrated as tolerated for 2-8 weeks to a dose that improved PAH symptoms. Investigators chose the frequency and dose increments for up-titration and were instructed to utilize parenteral therapy to achieve an optimal treprostinil dose; titration was individualized and optimized in each participant to a dose that improved PAH symptomology. There was no maximum parenteral treprostinil dose.

Once patients reached at least 20 ng/kg/min and were deemed suitable for transition based on physician assessment, they were transitioned at Week 2,4, or 8 to oral treprostinil via cross-titration in an inpatient or outpatient setting over 1-21 days. All participants still receiving parenteral treprostinil at Week 8 began transitioning to oral treprostinil regardless of their parenteral treprostinil dose unless deemed unsuitable for transition by their clinician. Before transition, 6MWD, WHO FC, NT-proBNP, and echocardiography parameters were assessed.

The oral treprostinil daily dose was calculated according to formula I. Dosing conversion steps and representative cross-titration for outpatient and inpatient transition are shown in Tables 2-3. A post-transition visit occurred 7-14 days after initiating oral treprostinil. Oral treprostinil was titrated to a maximum tolerated dose up to Week 16. Clinicians were encouraged to continue oral treprostinil titration in an outpatient setting by increasing the dose by 0.125 mg TID every 3-4 days as tolerated by the patient. The target dose of oral treprostinil at the end of transition was determined using the following weight-based equation: (0.0072)×(patient weight in kg)×(parenteral dose in ng/kg/min).

TABLE 2
Representative outpatient transition dosing schedule.

| | | Parenteral | Oral | Oral |
|---|---|---|---|---|
| | | Treprostinil: | Treprostinil: | Treprostinil: |
| Transition | Time | Dose | Single Dose | Total Daily |
| Day | of Day | (ng/kg/min) | (mg) | Dose (mg) |
| Day 1 | Morning | 26 | 0 | 1 |
| (0-24 hours) | Afternoon | 24 | 0.375 | |
| | Evening | 22 | 0.625 | |
| Day 2 | Morning | 21 | 1 | 4 |
| (24-48 hours) | Afternoon | 19 | 1.375 | |
| | Evening | 17 | 1.625 | |
| Day 3 | Morning | 16 | 2 | 7 |
| (48-72 hours) | Afternoon | 14 | 2.375 | |
| | Evening | 12 | 2.625 | |
| Day 4 | Morning | 10 | 3 | 10 |
| (72-96 hours) | Afternoon | 8 | 3.375 | |
| | Evening | 6 | 3.625 | |
| Day 5 | Morning | 4 | 4 | 12.625 |
| (96-120 hours) | Afternoon | 2 | 4.25 | |
| | Evening | 0 | 4.375 | |

TABLE 3
Representative inpatient transition dosing schedule.

| | | Parenteral | Oral | Oral |
|---|---|---|---|---|
| | | Treprostinil: | Treprostinil: | Treprostinil: |
| Transition | Time | Dose | Single Dose | Total Daily |
| Day | of Day | (ng/kg/min) | (mg) | Dose (mg) |
| Day 1 | Morning | 30 | 0 | 6 |
| (0-24 hours) | Afternoon | 15 | 2 | |
| | evening | 0 | 4 | |

Endpoint Measurements

The primary endpoint was the percentage of patients who achieved an oral treprostinil TDD of at least 12 mg (0.171 mg/kg for patients <70 kg) at Week 16. Secondary endpoints included change from baseline to Week 16 in risk strata and clinical parameters, where baseline values were collected within 14 days prior to parenteral treprostinil initiation. Changes in risk strata for FC, NT-proBNP, 6MWD, and right atrial (RA) area were assessed using the 2015 ESC/ERS Guidelines, which categorizes risk for each determinant as low, intermediate, and high (Galiè et al., 2016). Clinical parameters of interest included FC, NT-proBNP, 6MWD, echocardiography parameters, REVEAL Lite 2 score (Benza et al., 2021), and Borg Dyspnea Score (Borg, 1982, DOI: https://doi.org/10.1249/00005768-198205000-00012). The REVEAL Lite 2 score includes clinical parameters to determine the risk status (low, intermediate, and high) for pulmonary arterial hypertension. The Borg Dyspnea Score ranges from 0 to 10, where 0 indicates no dyspnea and 10 indicates very, very heavy (almost maximal) dyspnea. Patient-reported outcomes were assessed using the emPHasis-10 quality of life questionnaire (Yorke et al., 2014, DOI: 10.1183/09031936.00127113) and the Pulmonary Arterial Hypertension Symptom Scale (PAHSS) (Matura et al., 2015, DOI: https://doi.org/10.1016/j.apnr.2014.04.001). The Treatment Satisfaction Questionnaire for Medication (TSQM) (Atkinson et al., 2004, DOI: https://doi.org/10.1186/1477-7525-2-12) was used to measure patient satisfaction with their medication over the last two to three weeks; higher scores indicate greater satisfaction. EmPHasis-10 scores can range from 0 to 50, where lower scores indicate better quality of life.

Safety and tolerability were assessed at each scheduled visit and in between visits as needed throughout the duration of the study. Tolerability measures of interest included incidence of all adverse events (AEs) and prostanoid-related AEs including headache, diarrhea, nausea, vomiting, flushing, jaw pain and extremity pain. A survey took measurements of severity and duration of AEs commonly associated with prostanoid therapy: headache, diarrhea, nausea, vomiting, flushing, jaw pain and extremity pain; scores range from 0 (not at all bothersome, zero days) to 14 (very bothersome, every day). Prostanoid-related events captured by the survey were only recorded if the event was unusual with respect to intensity, frequency, or duration compared to symptoms in the patient's medical history. Patient compliance with taking oral treprostinil was assessed by performing study drug accountability at each scheduled study visit.

Statistics

The primary endpoint and all efficacy endpoints were analyzed using the per-protocol population, which included all patients without major protocol deviations unless otherwise specified. The exact (Clopper-Pearson) 95% confidence interval was calculated for the primary endpoint. Safety and tolerability were analyzed using the safety population, which included all patients who initiated parenteral treprostinil. For continuous variables, descriptive statistics are reported as median (interquartile range, IQR or range) or mean (±standard deviation, SD). For categorical variables, descriptive statistics include frequency and percentage of patients in each category. Changes from baseline to Week 16 for continuous variables were analyzed using Wilcoxon signed rank test. The change in FC from baseline to Week 16 was analyzed using McNemar's test. The p-values from these tests were derived for descriptive purposes and not part of a formal hypothesis testing framework.

The institutional review board for human studies approved the protocols and written consent was obtained from the subjects or their surrogates if required by the institutional review board.

### Results

Primary Endpoint Measures

Thirty-five patients began the study. Twenty-nine of these patients received an initial dose of oral treprostinil, and 28 patients completed the study and received oral treprostinil at Week 16. Patients were on parenteral treprostinil for a mean duration of 55 days (±13) and reached a mean parenteral dose of 27.0 ng/kg/min (±9.6) at the time of transition to oral treprostinil. The median (range) dose immediately before transition was 24 (6-40) ng/kg/min. In the study, the maximum parenteral doses achieved during initiation and up-titration were similar between participants who began transition to oral treprostinil at Week 4 compared to Week 8, suggesting 4 weeks or less may be an adequate time to up-titrate patients to therapeutic SC or IV doses.

During the transition visit (Week 2, 4, or 8), participants transitioned if they achieved a minimum parenteral treprostinil dose of 20 ng/kg/min and were deemed suitable for transition based on physician assessment. During the transition visit (Week 2, 4, or 8), participants transitioned if they achieved a minimum parenteral treprostinil dose of 20 ng/kg/min.

Transition from parenteral to oral treprostinil could occur over 1-21 days in an inpatient or outpatient setting. Over half of the patients (55%) transitioned to oral treprostinil in the outpatient setting over a mean duration of 5.6 days (±2.3). The remaining 45% of patients transitioned in the inpatient setting over a mean duration of 1.7 days (±0.5). A post-transition visit took place one to two weeks after transition was initiated. The mean (SD) TDD at the post-transition visit was 16.6 (8.1) mg. At the end of the study, 79.3% of participants achieved an oral treprostinil dose of at least 12 mg TDD at Week 16; the mean (SD) TDD was 16.4 (7.5) mg and the median dose was 15.0 mg (IQR 12.0, 22.9) at Week 16. The mean (SD) oral treprostinil exposure time was 64 (16) days throughout the entire study.

From the post-transition visit to Week 16, many participants (13 of 28) continued to up-titrate oral treprostinil after their posttransition visit, while nine participants maintained and six participants decreased their oral treprostinil dose. No participants switched back to parenteral treprostinil after receiving oral treprostinil.

While participants were on parenteral treprostinil, 69%, 55%, and 34% of participants in the per-protocol population (n=29) received ondansetron, acetaminophen, and loperamide for prostacyclin-related nausea/vomiting, headache, and diarrhea, respectively. When on oral treprostinil, 48%, 38%, and 38% of participants used ondansetron, acetaminophen, and loperamide, respectively. Overall, the use of all concomitant medications decreased after transitioning from parenteral to oral treprostinil.

Secondary Outcome Measures

Adverse Events

All patients who initiated parenteral treprostinil reported at least one treatment-emergent AE. Table 4 shows the AEs experienced by patients during the parenteral treprostinil phase, the transition phase, and the oral treprostinil phase. Adverse effects not included in Table 4, but occurring in at least 10% of the patients, include decreased appetite, joint pain, dyspepsia, abdominal pain, infusion site irritation, back pain, dyspnea, muscle spasms, peripheral edema, and dizziness. During the parenteral treprostinil induction, the most bothersome AE was extremity pain, followed by jaw pain, headache, and diarrhea; all except diarrhea improved to ‘not at all bothersome,’ on a scale for present adverse effects where 1 equals ‘bothers me a lot’ and 4 equals ‘not at all bothersome,’ after transitioning to oral treprostinil. Following transitioning to oral treprostinil, diarrhea and nausea became the most bothersome AEs. The number of patients experiencing prostanoid-related AEs was similar during the parenteral and oral treprostinil phases, except for an increase in flushing and a decrease in headache after transition.

TABLE 4
Prostanoid-related adverse events. Presented are the
number and associated percentage of patients who experienced
a specific AE during each phase of the study.

| | Parenteral | | Oral |
|---|---|---|---|
| | Treprostinil | | Treprostinil |
| | Phase (Before | Transition | Phase (After |
| | Transition) | Phase | Transition) |
| | n = 35 | n = 33 | n = 32 |
| | Adverse Events, n (%) | | |
| Headache | 28 (80) | 19 (58) | 19 (59) |
| Vomiting | 13 (37) | 5 (15) | 9 (28) |
| Nausea | 24 (69) | 21 (64) | 21 (66) |
| Extremity pain | 12 (34) | 10 (30) | 11 (34) |
| Diarrhea | 18 (51) | 17 (52) | 17 (53) |
| Jaw pain | 16 (46) | 15 (46) | 16 (50) |
| Flushing | 10 (29) | 10 (30) | 17 (53) |

Clinical Parameters and Risk Assessment

At the transition visit, the median (IQR) of 6MWD, NT-proBNP, tricuspid annular plane systolic excursion (TAPSE), and right atrial area for the per-protocol population were 377 (318, 453) m, 186 (110, 724) ng/L, 18.1 (14.8, 20.9) mm, and 19.3 (16.0, 26.7) cm2, respectively. Of the 29 patients, 76% improved WHO FC, 21% maintained, and 3% worsened at their transition visit. In general, the clinical variables outlined above improved from baseline to transition (Table 1).

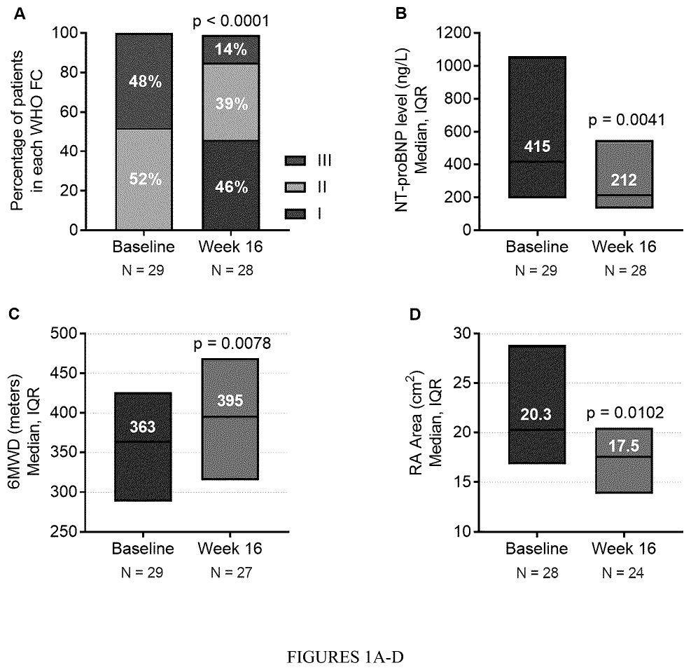

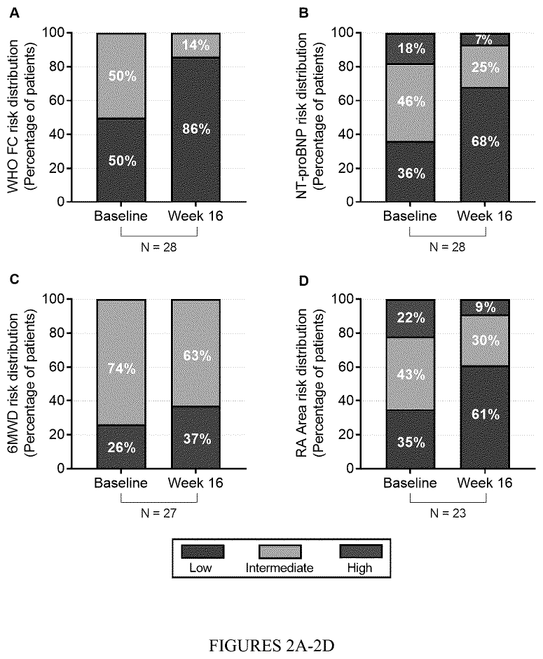

At Week 16, multiple clinical measurements were improved from the baseline measurements (Tables 1 and 5, FIGS. 1 and 2). Patients demonstrated clinical improvements in echocardiography parameters. From baseline to Week 16, the median change in RA area was −2.9 cm² (IQR −6.6, 1.5; p=0.0102), reaching a median RA area of 17.5 cm² (IQR 13.8, 20.5) at Week 16 (FIG. 1D). RA area was determined using end diastolic area. In addition to RA area, several other echocardiography parameters had numerically favorable changes. Table 5 shows changes in echocardiography parameters for patients in the study. The median change from baseline to Week 16 in cardiac output was +0.5 L/min (IQR −0.4, 1.4), median change in tricuspid annular plane systolic excursion (TAPSE) was +1.2 mm (IQR −1.6, 4.2), and median change in left ventricular diameter was +3.5 mm (IQR −0.7, 6.4).

TABLE 5
Changes in echocardiography parameters. Values shown are median (IQR). TAPSE:
tricuspid annular plane systolic excursion; RA: right atrium; RV: right ventricle;
MPI: Myocardial performance index; LV: left ventricle; LVOT: left ventricular
outflow tract; PVAT: pulmonary valve acceleration time.

| | | | | | Change from | | |
|---|---|---|---|---|---|---|---|
| Parameter | Baseline | | Week 16 | | Baseline | | P-value |
| Heart rate (bpm) | 73 | (68; 83) | 76 | (69; 82) | +2 | (−4; 5) | 0.6990 |
| Cardiac output | 4.4 | (3.5; 5.1) | 4.9 | (4.2; 6.0) | +0.5 | (−0.4; 1.4) | 0.1173 |
| (L/min) | | | | | | | |
| TAPSE (mm) | 17.0 | (12.4; 21.2) | 19.9 | (14.9; 22.1) | +1.2 | (−1.6; 4.2) | 0.0820 |
| RA area (cm²) | 20.3 | (16.8; 28.9) | 17.5 | (13.8; 20.5) | −2.9 | (−6.6; 1.5) | 0.0102 |
| RV diameter (mm) | 32.7 | (24.9; 36.1) | 24.8 | (20.6; 32.2) | −2.6 | (−9.2; 1.2) | 0.1231 |
| RV wall strain (%) | −21.8 | (−24.7; −15.0) | −19.2 | (−23.8; −15.9) | +1.3 | (−2.4; 3.0) | 0.7019 |
| RV MPI | 0.5 | (0.4; 0.7) | 0.4 | (0.3; 0.7) | −0.1 | (−0.1; 0.1) | 0.1643 |
| RV/LV ratio, | 0.8 | (0.6; 0.9) | 0.6 | (0.5; 0.7) | −0.1 | (−0.2; 0.0) | 0.0583 |
| diastole | | | | | | | |
| LVOT (mm) | 20.1 | (17.9; 21.1) | 19.6 | (19.2; 21.2) | +0.4 | (−0.2; 1.3) | 0.0249 |
| LVOT integral | 21.1 | (16.1; 23.2) | 19.5 | (17.8; 24.2) | +0.9 | (−3.5; 3.8) | 0.6261 |
| (cm) | | | | | | | |
| LV diameter (mm) | 41.7 | (39.1; 43.4) | 44.9 | (41.6; 49.3) | +3.5 | (−0.7; 6.4) | 0.0103 |
| LV eccentricity, | 1.0 | (1.0; 1.1) | 1.0 | (1.0; 1.1) | 0.0 | (−0.1; 0.1) | 0.2584 |
| diast. | | | | | | | |
| LV eccentricity, | 1.3 | (1.1; 1.5) | 1.2 | (1.0; 1.5) | +0.1 | (−0.2; 0.2) | 0.4780 |
| syst. | | | | | | | |
| PVAT (ms) | 83.4 | (76.9; 98.9) | 89.9 | (79.1; 99.6) | +4.4 | (−5.7; 18.5) | 0.1594 |

The WHO-FC system has functional classes I-IV where a lower FC (e.g. I) indicates less severe disease. There was an overall shift towards less severe FC symptoms from baseline to Week 16, with 68% of patients improving in FC and only one patient worsening (p<0.0001). From baseline to Week 16, the percentage of patients classified as FC I increased from 0% to 46% (FIG. 1A).

Patients demonstrated clinical improvements in 6MWD. The median increased from 363 m (IQR 288, 426) at baseline to 395 m (IQR 315, 469) at Week 16 (Table 1, FIG. 1C). The median Borg Dyspnea Score improved from 4 (IQR 3, 6) to 3 (IQR 1, 4) at Week 16, with a median change from baseline of −1 (IQR −3, 0; p=0.0009). From baseline to Week 16, improvements were seen in most domains of the PAHSS including fatigue, dyspnea, edema, orthopnea, and dizziness; there was little change in chest pain and syncope. Quality of life significantly improved, as measured by a median change from baseline in emPHasis-scores of −3 (p=0.0001). Median NT-proBNP at Week 16 was 212 ng/L (IQR 132, 551) with a median change from baseline of −134 ng/L (IQR −360, 1.5; p=0.0041) (FIG. 1B).

From baseline to Week 16, the median REVEAL Lite 2 score improved from 6 (IQR 4, 7) to 3.5 (IQR 2, 5.5), with a median change from baseline to Week 16 of −1 (IQR −3, 0; p=0.0006).

Improvements were also seen in the 2015 ESC/ERS risk stratification for FC, 6MWD, NT-proBNP, and RA area (FIG. 2A-2D). The percentage of patients in the low-risk strata for all four variables increased at Week 16, most notably for NT-proBNP and RA area, where the percentage of patients in the low-risk strata almost doubled. Accordingly, the percentage of patients in the intermediate and high-risk strata decreased at Week 16. From baseline to Week 16, the percentage of patients who shifted to a lower risk stratum for FC, NT-proBNP, 6MWD, and RA area was 39%, 39%, 15%, and 35%, respectively. There was only one patient whose risk stratification worsened for each variable.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above.

All numerical designations, e.g., pH, temperature, time, concentration, amounts, and molecular weight, including ranges, are approximations which are varied (+) or (−) by 10%, 1%, or 0.1%, as appropriate. It is to be understood, although not always explicitly stated, that all numerical designations may be preceded by the term “about.” It is also to be understood, although not always explicitly stated, that the reagents described herein are merely exemplary and that equivalents of such are known in the art.

All patents, patent applications, provisional applications, and publications referred to or cited herein are incorporated by reference in their entirety, including all figures and tables, to the extent they are not inconsistent with the explicit teachings of this specification.

What is claimed is:

1. A method for treating pulmonary hypertension comprising administering to a subject suffering from pulmonary hypertension a first therapeutically effective amount of a non-oral therapeutic agent for treating pulmonary hypertension followed by an orally administered therapeutic agent for treating pulmonary hypertension, wherein the first therapeutically effective amount of non-oral therapeutic agent is sufficient to allow for an increased amount of the orally administered therapeutic agent to thereafter be administered compared to a subject who was not previously treated with the non-oral therapeutic agent.

2. The method of claim 1, wherein the first therapeutically effective amount of the non-oral therapeutic agent is a parental prostacyclin.

3. The method of claim 2, wherein the parenteral prostacyclin is parenteral treprostinil, a salt, an ester or a prodrug thereof.

4. The method of claim 3, wherein the parenteral treprostinil is intravenous treprostinil.

5. The method of claim 3, wherein the parenteral treprostinil is subcutaneous treprostinil.

6. The method of claim 3, wherein the parenteral treprostinil is initiated at a dose of 2 ng/kg/min.

7. The method of claim 3, wherein the parenteral treprostinil is titrated to a minimum dose of 20 ng/kg/min over at least two weeks.

8. The method of claim 3, wherein the parenteral treprostinil is titrated to a minimum dose of 20 ng/kg/min over a period from about two weeks to about eight weeks.

9. The method of claim 1, wherein the orally administered therapeutic agent is an orally administered form of treprostinil, a salt, an ester or a prodrug thereof.

10. The method of claim 9, wherein the orally administered form of treprostinil is administered two times per day.

11. The method of claim 9, wherein the orally administered form of treprostinil is administered three times per day.

12. The method of claim 9, wherein the orally administered form of treprostinil is titrated up to at least 12 mg per day from an initial orally administered dose of 1 mg per day over a period of up to 21 days.

13. The method of claim 9, wherein the orally administered form of treprostinil is titrated up to at least 12 mg per day from an initial orally administered dose of 0.5 mg per day over a period of up to 21 days.

14. The method of claim 1, wherein at least one side effect selected from the group consisting of headache, nausea, and vomiting improves after transition to the orally administered therapeutic agent.

15. The method of claim 1, wherein the area of the subject's right atrium decreases.

16. The method of claim 1, wherein the subject's six-minute walk distance increases.

17. The method of claim 1, wherein the pulmonary hypertension is pulmonary arterial hypertension.

18. The method of claim 1, wherein the method comprises treating the subject with additional therapeutic agents.

19. The method of claim 18, wherein the additional therapeutic agents treat pulmonary hypertension.

20. The method of claim 18, the additional therapeutic agents comprising endothelin receptor antagonists, phosphodiesterase-5 inhibitor, and soluble guanylate cyclase stimulator.

21. The method of claim 1, wherein the orally administered therapeutic agent is selected from the group consisting of treprostinil, beraprost, selexipag and ralinepag.

22. The method of claim 1, wherein the therapeutically effective amount of the non-oral therapeutic agent is an inhaled prostacyclin.

23. The method of claim 22, wherein the inhaled prostacyclin is inhaled treprostinil.
